(19) United States
(12) Patent Application Publication
(10) Pub. No.: US 2026/0306863 A1
(43) Pub. Date: Oct. 8, 2026

(54) METHOD AND SYSTEM FOR AFFECTING PERMEABILITY OF CRANIAL BLOOD-ARACHNOID BARRIER

(71) Applicant: SHEBA IMPACT LTD., Ramat-Gan (IL)

(72) Inventors: Yael MARDOR, Hetzeliya (IL); Shirley SHARABI, Ganei Tikva (IL); David LAST, Jerusalem (IL); Itzik COOPER, Rehovot (IL); Dianne DANIELS, Ramat Hasharon (IL)

(73) Assignee: SHEBA IMPACT LTD., Ramat-Gan (IL)

(21) Appl. No.: 19/482,156

(22) PCT Filed: May 9, 2024
(86) PCT No.: PCT/IL2024/050455

§ 371 Date: Nov. 6, 2025

Related U.S. Application Data
(60) Provisional application No. 63/501,031, filed on May 9, 2023.

(51) Int. Cl. A61N 1/40; A61N 1/04; G16H 20/30

(52) U.S. Cl. CPC A61N 1/40; A61N 1/0428; A61N 1/0476; A61N 1/0484; G16H 20/30

(57) ABSTRACT

A blood-arachnoid barrier (BAB) disruption system is presented comprising: a head wearable support structure defining a plurality of electrode sites for a plurality of electrodes of an electrodes' arrangement comprising independently operable electrode unit(s) generating an electric field; and an electronic unit comprising electric pulse generator(s) and a controller for managing operation of the electrode unit(s) to generate the electric field. The electrode unit comprises the electrodes of selected optimized geometrical and position parameters, and the controller is adapted to manage the operation of the electrode unit(s) to generate a pulsed electric field of selected optimized electric parameters. The optimization is such that the electric field within a target BAB region has a characteristic field profile and a characteristic field value being above a predetermined BAB disruption threshold for a given pulse duration, and satisfies a condition of a predetermined optimized AB-ratio between percentage of the target BAB region exposed to the electric field above said BAB threshold and percentage of brain tissues exposed to the electric field of a value above a predetermined brain threshold for the given pulse duration, thereby avoiding BBB disruption by said electric field.

### TECHNOLOGICAL FIELD

The present invention is in the field of medical applications and relates to a system and method for affecting permeability of cranial blood-arachnoid barrier by application of extracranial pulsed electric fields.

### BACKGROUND

The central nervous system (CNS) is protected against harmful substances contained in the blood by the blood-brain barrier (BBB) and the blood-cerebrospinal barrier (BCSFB). The BCSFB at the choroid plexus is formed by epithelial cells and have continuous intercellular tight junctions (TJs) and adherens junctions (AJ) that lack fenestrations. TJs are composed of a branching network of sealing strands, each strand being formed from a row of transmembrane proteins embedded in both plasma membranes, with extracellular domains joining one another directly. Major protein types include claudins and occludins. An AJ is defined as a cell junction whose cytoplasmic face is linked to the actin cytoskeleton. They can appear as bands encircling the cell (zonula adherens) or as spots of attachment to the extracellular matrix (adhesion plaques). On the extracellular surface, AJs are typically composed of cadherins, a family of transmembrane proteins that form homodimers in a calcium-dependent manner with other cadherin molecules on adjacent cells. As such, the epithelium of the BCSFB acts as a ‘physical barrier’ limiting paracellular movement of molecules and a gate controlling molecular traffic into and out of CSF. The other part of the BCSFB is the arachnoid membrane, which envelops the brain. The cells of this membrane are also linked by TJ and AJ. The barrier at the arachnoid membrane is referred to as blood-arachnoid barrier (BAB).

The subarachnoid space, where cerebrospinal fluid (CSF) flows over the brain and spinal cord, is lined on one side by BAB cells. The BAB actively limits the transport of substances from the peripheral circulation into the CSF. As about 85% of the CSF is located in the subarachnoid space, it is the most significant limitation of drug delivery into the CSF.

Leptomeningeal diseases, including leptomeningeal metastases, are therefore extremely hard to treat. The main treatment option today is intrathecal delivery of therapeutic agents. Nevertheless, the treatment has significant limitations. When drug is injected into the CSF compartment, the drug clears relatively rapidly into the peripheral blood across the arachnoid villi. Thus, it is necessary to administer high drug concentrations into the CSF compartment and the ependymal surface is exposed to very high drug concentrations, which can have toxic side effects [Ghazal Naseri Kouzehgarani, et al., Harnessing cerebrospinal fluid circulation for drug delivery to brain tissues, Advanced Drug Delivery Reviews, Volume 173, June 2021, Pages 20-59]. This is one of the reasons why only a limited number of drugs are approved for intrathecal administration. Additional limitations are the invasiveness of the treatment and obstructions caused by the disease. Intrathecal delivery is usually administered intraventricular or into the lower spinal region where CSF pockets enable insertion of catheters. Intrathecal delivery is usually not performed directly into the sub-arachnoid space due to its narrow structure. Drugs delivered into these CSF pocket regions may not reach the subarachnoid region efficiently due to dilution and obstructions.

### GENERAL DESCRIPTION

As described above, the conventional approach of drug delivery into the CSF region imposes limitations on the types and doses of drugs that can be administered. Also, the conventional approach is typically invasive requiring placement of catheters/pumps.

If the blood-arachnoid barrier either in the cranium or in the spine would be transiently disrupted, therapeutic agents in the blood system (delivered intravenously (IV), orally or any other way), could pass from the blood stream directly into the CSF in the subarachnoid space. This would allow application of subtle disruption, thus maintaining a relatively low, less-likely to be toxic, drug concentration in the CSF.

Thus, transiently disrupting the BAB, to enable therapeutic agents penetration into the CSF, can be beneficial for patients with meningeal or CNS diseases. Partial list of indications includes: Leptomeningeal tumors (Melanoma, breast cancer, lung cancer, ovarian cancer, CNS lymphoma, Acute lymphocytic leukemia (ALL), Von Hippel-Lindau (VHL), Multiple sclerosis, Bacterial meningitis, Extraction/clearance of substance from the CSF—for example Amyloids in Alzheimer's disease, waste products, etc, High intracranial pressure (ICP), Unresponsive Cluster headaches or trigeminal neuralgia.

Transiently disrupting the BAB and increasing the concentration of certain drugs or molecules in the CSF can also increase the distribution of those molecules in the brain (as in the case of antisense oligonucleotides-ASOs).

The present disclosure provides a novel technique for obtaining transient BAB disruption (BABd) in significant BAB portions by inducing extracranial electric fields over a given threshold on the BAB while, as much as possible, sparing the brain from exposure to the electric fields and reducing treatment time. The technique of the present disclosure provides for optimized BAB disruption by applying the electric field of an optimal electric field profile and value to the arachnoid region while minimizing the electric field application to the brain parenchyma region.

The above is achieved by optimizing such parameter as a brain-arachnoid ratio (AB-ratio) being a ratio between the percentage of a BAB region exposure to the electric field above certain BAB threshold and the percentage of brain tissues exposure to electric field inducing nerve stimulation (i.e., electric field above the brain threshold), thereby providing effective BAB region disruption while avoiding BBB disruption by said electric field.

Thus, the optimized structure and operation of the electrodes' arrangement is such that the electric field produced by the electrode unit is over the threshold for inducing BAB disruption in a portion of the BAB, and the maximum strength of the electric field in the brain is below the threshold for inducing blood brain barrier (BBB) disruption.

It should be understood that the BAB threshold value, as well as the brain threshold value, depends on the duration of the pulsed electric field application. For example, the brain threshold is approximately 0.2 V/cm for electric pulses with a duration of 300 us and 0.5 V/cm for pulse duration of 50 μs. The BAB threshold is for example 30-35 V/cm for electric pulses with a duration of 50 μs.

The AB-ratio is calculated as: AB-ratio=% BABd/% brain, where % ABd relates to the percentage of BAB area being disrupted (exposed to electric fields above the BAB disruption threshold) and % brain relates to the percentage of brain volume exposed to electric field above neuron-excitation threshold. The optimal AB-ratio is at least about 0.1. It should be understood that providing such a ratio is challenging because both BAB disruption and brain neurons' stimulation are non-linearly dependent on the applied voltage/current.

The technique of the present disclosure enables disrupting a pre-determined surface area. The size and location of the disruption can be determined pre-treatment using a personal treatment planning based on imaging such as MRI or CT and patient-based data.

The technique of the present disclosure enables choosing the disruption location, thus avoiding specific areas if desired or targeting others. For example, it may be desired to disrupt a portion of the BAB that is close to a tumor or leptomeningeal metastases or avoid applying electric fields in undesired portion of the BAB such as near areas where the CSF flow is obstructed. Furthermore, the size of the disruption region/area can also be determined based on the desired drug concentration in said region/area, the patient's previous treatments and clinical condition.

The technique of the present disclosure provides BABd by utilizing application of electric fields while significantly reducing electric fields penetration into the brain region. In other words, the technique of the present disclosure utilizes a “surface treatment”, applied to the arachnoid region (outer surface of the brain), being a fully non-invasive treatment and providing effective BABd.

The above is achieved by optimizing the configuration and operation of an electrodes' assembly/arrangement, i.e. geometrical and electric parameters of each electrode unit, as well as position parameters of the electrode unit with respect to the BAB region, and position parameters of electrodes of the electrode unit. The optimization procedure is preferably a personalized procedure utilizing analysis of patient-related data, which may be extracted from static image data such as MRI or CT.

More specifically, a system configured and operable according to the present disclosure comprises an electronic unit including a controller (programmed microcontroller) and pulse generator(s) (associated with current or voltage source(s)) connected to a plurality of uncoupled electrodes, i.e., separately/independently operable electrodes of each of one or more electrode units. The electronic unit operates the electrodes of predetermined optimized geometrical and position parameters, according to a predetermined optimized operational pattern (e.g., sequences that can be synchronous, asynchronous or sequential), to thereby produce the electric field of the desired optimized profile and value.

The electrodes are operated such that each electrodes unit delivers pre-defined pulse trains. As noted above, preferably, the operational parameters/conditions are established per patient, according to a personal treatment planning that will allow protecting specific regions in the brain (such as the brain stem or the hippocampus) from exposure to electrical field.

The optimization, aimed at providing the BAB disruption by applied electric fields while significantly reducing (minimizing) electric fields penetration into the brain region, may include selection of such parameters of the electrodes' arrangement as a size (diameter) of the electrode, distance between the centers of the electrodes of the same electrode unit, number of electrodes in the electrode units, location of the electrode unit(s), and the applied current/voltage.

Thus, in some embodiments, the optimization procedure is personalized, i.e., is a patient-specific (or may be population-specific). The patient-related data can be extracted from static image data such as MRI or CT, and used to define the corresponding optimal treatment parameters/conditions. These include operational data (electric data) and electrodes' array design data (sizes and relative accommodation of the electrodes to be involved in the treatment session). The treatment parameters define the optimal AB-ratio provided by the electric field produced by the optimally configured and operated electrodes' arrangement.

As will be described further below, the inventors have shown that, while for a given size of electrodes, the smallest distance between the electrodes of the electrode unit provides the largest AB-ratio, larger distances can be used for lower currents (larger distance between the electrodes allows increased penetration which may compensate for lower penetration caused by the lower currents).

As noted above, the system of the present disclosure includes the electrodes' arrangement including a plurality of electrodes that can be operated/activated to form a number of electrode units of uncoupled electrodes, connected to the pulse generator(s) (current or voltage source(s)).

Each electrode unit includes at least two electrodes, which may form: an electrode pair; one positive electrode and two negative electrodes, several positive and negative electrodes, etc. The terms “coupled electrodes” or “uncoupled electrodes” refers to the electrical connection between the different electrode units. Each unit is insulated from the other unit(s).

In some embodiments, the technique of the present disclosure utilizes a pattern of pulsed operation of each electrode unit with a predetermined (low) duty cycle, such that the pulse duration produced by the electrode unit is (significantly) smaller than the time period between the successive pulses generated by said electrode unit. For example, a treatment given at 1-4 Hz may use pulse duration of 1-200 μsec. This allows operation of the electrodes' arrangement, including a plurality of electrode units, with a sequence of pulses of different electrode units within the duty cycle, and accordingly enables to simultaneously activate multiple electrode units within a larger total surface area of treatment.

Each electrode unit may include one grounded electrode and one energized electrode, several grounded electrodes coupled with a single energized electrode, a single grounded electrode with several energized electrodes, or may include several grounded and energized electrodes in different arrays. The energized electrodes may be connected to one electric source or can be connected to multiple separate sources.

Alternatively, each electrode unit may be a coil, and the generator(s) includes a changing electromagnetic source activating the coils in order to produce the electric field, having electromagnetic field components, on the BAB being sufficient for inducing BAB disruption.

As noted above, in some embodiment of the present disclosure the treatment parameters are established per patient, according to a personal treatment planning based on personal properties such as skin and skull thickness in the desired treatment area or disease structures. For example, the amplitude on each couple of electrodes may be set according to the thickness of the skull near the electrodes location, so that in regions where the skull is thinner, lower amplitudes are chosen in order to get similar exposure to electric field as regions where the skull is thicker. Alternatively, the treatment plan can be one of several pre-defined programs, chosen based on imaging criteria or disease types.

In some embodiments the location of the electrodes is set in an attempt to avoid non-intact regions in the skull (such as post op regions) in order to avoid penetration of high electric fields into the brain.

In some embodiments, the electrode configuration, i.e., the size of the electrodes, the distance between them, and the current are chosen in a manner enabling maximal BAB exposure and minimal brain exposure to electric fields that will allow protecting specific regions (such as the brain stem or the hippocampus) from exposure to high electrical field. In some embodiments the treatment plan and electrodes configuration are chosen to minimize the volume of the brain that will be stimulated synchronously.

In some embodiment of the present disclosure, the location of the electrode is further from arachnoid villi or granulations, through which the CSF exits the subarachnoid space and enters the general circulation. Additionally, the electrodes are further from the cribriform plate or cranial and spinal nerves to minimize lymphatic drainage outside the CNS.

In some embodiments, only one unit may be activated per treatment or 10 couples or 100 couples or 1000 couples. The electrodes units may be activated simultaneously, or serially, or in a synchronized manner with a phase delay manner, in which the phase delay for each electrode couple is different and is smaller than the interval between pulses.

In some embodiments, each pulse train comprises a single pulse or a train between 1 and 1000 pulses. For example, each pulse train can be applied once per treatment or repeated at least 2 times, 10 times or 100 times.

In some embodiments, each pulse in the pulse train is a sinusoidal, triangular or rectangular pulse, or a Gaussian pulse.

In some embodiments, each pulse train is applied at a frequency of less than 100 Hz, less than 5 Hz, about 1 Hz, or less than 1 Hz. For example, the frequency of each pulse train is at least 0.5 Hz.

For example, each pulse duration is at least 10 nsec or at least 10 μs or 50 μs or 300 μs.

In some embodiments the electrodes are plate electrodes, or the electrodes are screw electrodes or the electrodes are needle electrodes or the electrodes are compromised from an array of micro electrodes or the electrodes are configured as coils.

In some embodiments the electrodes' diameter is 0.5 cm or it is 1 cm or 2 cm or it is less than 5 cm. Alternatively, the diameter of the array of micro electrodes is 0.5 cm or it is 1 cm or 2 cm or it is less than 5 cm.

In some embodiments the distance between the electrodes in each unit is smaller than 0.4 cm, smaller than 1 cm, smaller than 5 cm or smaller than 10 cm.

In some embodiments of the present disclosure, the treatment planning is AI-based technique, or an optimization algorithm based technique. The inputs of the program (software) may include but are not limited to: Patient clinical data, imaging data (MRI or CT based including tissue segmentation, skull/skin thickness, skull abnormalities/surgeries, regions of interest, arachnoid regions to avoid, brain regions to avoid), pharmacokinetics data (drug type, drug maximal dose, drug desired dose). The outputs of the optimization program may comprise a set of design instruction for personal electrode configuration and a set of activation instruction for the generators controller.

Thus, according to one broad aspect of the present disclosure, it provides a blood-arachnoid barrier (BAB) disruption system comprising:
a head wearable support defining a plurality of electrode sites for a plurality of electrodes of an electrodes' arrangement, said electrodes arrangement comprising at least one independently operable electrode unit configured and operable to generate an electric field; and an electronic unit comprising a signal generator and a controller configured and operable for managing operation of each of said at least one electrode unit to generate the electric field,
wherein each of said at least one electrode unit comprises the electrodes of selected optimized geometrical and position parameters, and the controller is configured and operable to manage the operation of each of said at least one electrode unit to generate the electric field being a pulsed electric field of selected optimized electric parameters, such that the electric field produced by the electrodes' arrangement within a target BAB region has a characteristic field profile and a characteristic field value being above a predetermined BAB disruption threshold for a given pulse duration, and satisfies a condition of an optimized AB-ratio, being a ratio between percentage of a BAB region exposed to the electric field above said BAB threshold and percentage of brain tissues exposed to the electric field of a value above a predetermined brain threshold for the given pulse duration, thereby avoiding BBB disruption by said electric field.

The optimized AB-ratio may be of about 0.04 or higher, e.g., may be 0.06 or 0.08 or 0.1.

The optimized geometrical and position parameters may comprise: electrode size for each electrode of the electrode unit, and a distance between centers of the electrodes of the electrode unit. The optimized geometrical and position parameters may comprise predetermined location for each electrode of said at least one electrode unit on the wearable support structure to thereby define optimal position of the electrode unit with respect to the target BAB region.

The electrodes' arrangement may comprise electrodes connectable to the same electric source of the electronic units and/or may comprise electrodes connectable to multiple separate sources of the electronic unit.

The electrodes' arrangement may include electrodes of any of the following types: microelectrodes array, surface electrodes, screw electrode or coils. For example the electrodes may be of a lateral size (diameter) in a range of 0.5 mm-1.5 cm.

The selected optimized electric parameters may comprise parameters of the pulsed electric field comprising amplitude, pulse width, pulse shape, pulse duration, number of pulses and frequency.

For example, the following electrical parameters can be used for each electrode unit: electric current 10 Amp-2 Amp (matching voltage depends on the impedance of the system and patient head) or voltage 10-2000V (matching current depends on the impedance of the system and patient head) with pulse duration of 500 ns-10 ms, number of pulses 1-1000 frequency of 0.5 Hz-1000 Hz. All on each unit of electrodes. The number of electrode unit may be properly selected from 1-1000 electrode units.

In some specific examples, the following electrical parameters can be used for each electrode unit from 1-20 electrode units: electric current of 0.4 Amp-1 Amp (matching voltage depends on the impedance of the system and patient head) or 400V-1000V matching current depends on the impedance of the system and patient head), pulse duration 1-100 micro second, number of pulses 10-500 frequency 0.5-50 Hz.

The electrodes' arrangement may be configured in accordance with an optimized treatment plan selected for a patient. Alternatively or additionally, the electrodes' arrangement may be defined by selected electrodes, from a plurality of electrodes mounted on said support structure, in accordance with an optimized treatment plan for a patient.

The electrodes' arrangement comprises a plurality of the independently operable electrode units, each forming a separately operable electric circuit.

The selected optimized electric parameters may comprise parameters defining electrodes' driving pattern. The driving pattern may correspond to synchronous, asynchronous or sequential activation of the electrodes.

The controller may be configured and operable to communicate with a control unit to receive therefrom personalized operational data comprising data indicative of the selected optimized geometrical and position parameters of the electrodes to be activated for BAB disruption session, and the selected optimized electric parameters of said electrodes to be activated. Alternatively or additionally, the controller may be configured and operable to analyze static image data of a patient's brain region and determine the optimized AB-ratio with respect to the target BAB region, and based on said optimized AB-ratio, generate personalized operational data comprising data indicative of the selected optimized geometrical and position parameters of the electrodes to be activated for BAB disruption session, and the selected optimized electric parameters of said electrodes to be activated.

According to another broad aspect of the present disclosure it provides a control unit configured and operable for managing BAB disruption treatment performed by electric field application, the control unit being configured as a computerized system comprising data input and output utilities and a data processor comprising an optimization utility configured and operable to analyze input data indicative of static image data of a patient's brain region, and determine personalized operational data comprising data indicative of selected optimized geometrical and position parameters of a plurality of electrodes to be activated during a BAB disruption treatment of a target BAB region and selected optimized electric parameters of said electrodes being activated to generate a pulsed electric field, such that the pulsed electric field produced by the electrodes' arrangement has a characteristic field profile and a characteristic field value being above a predetermined BAB disruption threshold for a given pulse duration of said pulsed electric field, and satisfies a condition of an optimized AB-ratio being a ratio between percentage of the target BAB region exposed to the electric field above said BAB threshold and percentage of brain tissues exposed to the electric field of a value above a predetermined brain threshold for the given pulse duration, thereby avoiding BBB disruption by said electric field.

As indicated above, the AB-ratio may be at least 0.04, e.g., may be 0.06 or 0.08 or 0.1 or higher.

The input data may comprise data indicative of MRI/CT data of a patient.

The input data may comprise at least one of the following: pharmacokinetics data of drugs intended to use, clinical data, and electrical data. The clinical data may comprise data indicative of a desired disruption area and undesired disruption areas. The electrical data may comprise at least one of the following parameters: current and voltage limitation, electric fields threshold, electric properties of head layers and boundary conditions.

The input data may comprise data indicative of one or more of the following: disruption area over the BAB, AB-ratio, areas where electric fields are to be avoided or minimized.

In some embodiments of the disclosure, the optimization further comprises administering a therapeutic agent to the subject in an amount sufficient to have the administered therapeutic agent be introduced into the bloodstream of the subject, wherein the treatment is capable of increasing permeability of the BAB for the therapeutic agent to be delivered into the CSF. The therapeutic agent may be delivered in close proximity, to the treatment, immediately prior to or post the treatment or at a later or earlier time point. Alternatively, the treatment may be applied without administering a therapeutic agent.

In some embodiments, the technique of the present disclosure can be further used to extract molecules from the CSF to the main circulation or lymphatic system. The treatment, in combination or without additional drugs can be used to clear or extract molecules such as waste products, amyloids, toxins, residual drugs, xenobiotic, drugs or immune system components.

In some embodiments, the technique is used in combination or without additional drugs to regulate CSF flow and intracranial pressure, for example by improving CSF clearance from the subarachnoid space.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 schematically exemplifies a BAB disruption system of the present disclosure;

FIG. 2A exemplifies a strength duration curve for transcranial magnetic stimulation;

FIGS. 2B and 2C show experimental results in a rat model demonstrating that below 700V (100 pulses, pulse duration of 50 μs at 4 Hz) no BBB disruption occurs and that the treatment is localized to the arachnoid barrier, wherein FIG. 2B shows examples of rat brains treated with PEFs at 600V and injected with Evans Blue dye, and FIG. 2C shows examples of rat brains treated with PEFs at 700V and injected with Evans Blue dye;

FIGS. 3A to 3C show experimental results in rat model demonstrating increased concentration of different molecular weight compounds in the CSF after application of PEFs (100 pulses, pulse duration of 50 μs at 4 Hz with different pulse amplitudes) for concentrations of Sodium Fluorescein (NaF)—FIG. 3A, Evans Blue (EB) tied to serum albumin—FIG. 3B, and Immunoglobulin G (IgG)—FIG. 3C;

FIGS. 4A and 4B show experimental results in rat model demonstrating voltage dependent effect of PEFs on the concentration of two mononuclear antibodies (mAb) based drugs, Nivolumab and Herceptin;

FIGS. 5A and 5B show PEFs treatment for BAB disruption enabling the penetration of significant amount of antisense oligonucleotides (ASOs) into mice brain from the CSF, for representative cortices samples of L-PEFs (FIG. 5A) and Sham treated mice (FIG. 5B);

FIG. 6 shows the reversibility of PEFs treatment: 4 hours post treatment the BAB is closed similar to the pre-treatment condition;

FIG. 7 shows the dependence on the drug dose;

FIG. 8 shows the dependence on electrodes size;

FIG. 9A shows geometrical model of the rats' head including electrodes location (6 mm radius at a distance of 30 mm) and layers constructed using Comsol Multiphysics (Comsol 5.3a, Stockholm, Sweden);

FIG. 9B shows one slice of the model with the layers (skin, skull, meninges, CSF and brain) and the electric conductivity of each layer;

FIGS. 10A-10F show the electric field distribution on the BAB (FIGS. 10A-10C) and brain surface (FIGS. 10D-10F) for different applied voltages (200V—FIGS. 10A and 10D; 600V—FIGS. 10B and 10E; and 700V—FIGS. 10C and 10F) for 6 mm radius electrodes at a distance of 30 mm;

FIGS. 11A and 11B show the segmentation of all cranial tissues based on MRI (FIG. 11A) and human atlas with the addition of the arachnoid layer (FIG. 11B);

FIG. 12 shows an example of an activation sequence of the electrodes;

FIG. 13A shows schematically an example of the parameters of electrodes' units, i.e., the diameter of the electrodes (φ cm), the distance between the centers of the electrodes (d cm) and the applied current (I mA), extracted from Realistic volumetric-Approach-based Simulator for Transcranial electric stimulation (Roast);

FIGS. 13B to 13E show the results of human simulation of the electric field distribution on the BAB) and brain parenchyma, wherein FIG. 13B shows the BAB area exposed to electric fields above 20 V/cm using 2 plate round electrodes with 1 cm diameter at 10 cm electrodes gap with applied current of 1 A; FIG. 13C shows the electric field distribution above stimulation level in the brain using the electrode configuration described in FIG. 13B; FIG. 13D shows the BAB area exposed to electric fields above 20 V/cm using 2 plate round electrodes with 1 cm diameter at 0.5 cm electrodes gap with applied current of 1 A; and FIG. 13E shows the electric field above stimulation level in the brain using the electrode configuration described in FIG. 13D;

FIG. 14A shows the AB-ratio-when the BAB disruption threshold is set to 20 V/cm and neuronal stimulation threshold is set to 0.5 V/cm as a function of electrode diameter for different distances between the electrodes' centers and different electrodes diameters for a given electric current of 1 Amp;

FIG. 14B shows the AB-ratio for a given current amplitude (1 Amp) and a given electrode size (1 cm) for different electrode gaps (d);

FIG. 14C shows the AB-ratio for different electrodes gaps (d) and applied currents (mA) with a given size of electrodes;

FIG. 15A schematically illustrates electrode configuration for applying electric fields to induce BAB disruption in a treatment region on the surface of the arachnoid of a subject; and

FIG. 15B shows an example of phased delay synchronous activation of electrodes units.

### DETAILED DESCRIPTION OF EMBODIMENTS

The BAB is an epithelial-like cell layer characterized by closely joined cells connected by an extensive and continuous system of tight junctions that separates and controls transport between the dura and the CSF filled subarachnoid space. The present disclosure presents the results of the inventors' study of the effects of low, extracranially applied, electrical (or electromagnetic) fields on the BAB of rats. The inventors have found that an increase in the permeability of the AB can occur even when BBB disruption doesn't occur in the brain parenchyma.

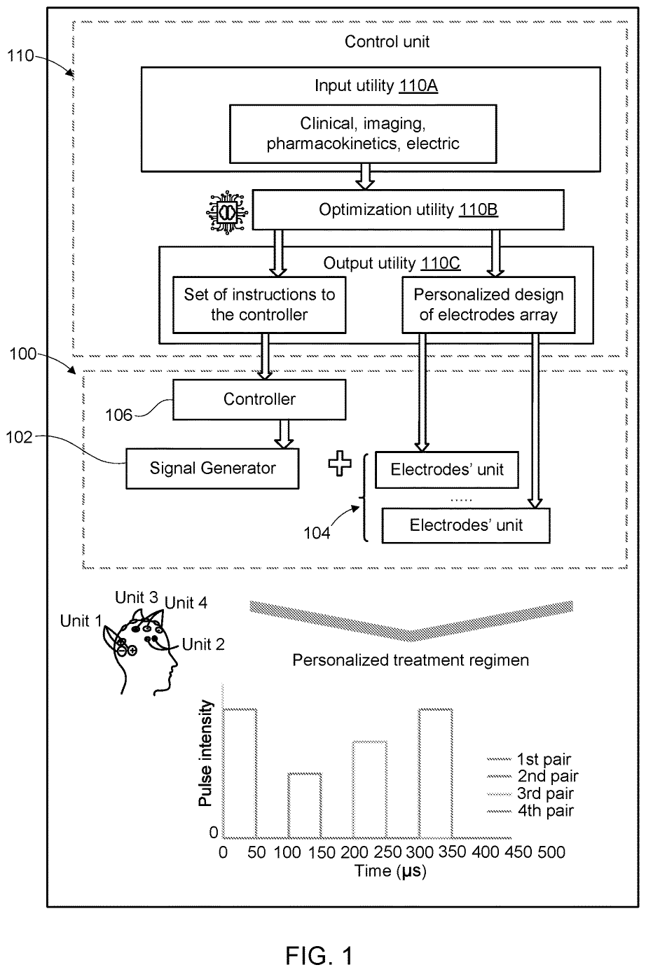

Reference is made to FIG. 1 illustrating schematically the BAB disruption system 100 of the present disclosure. The system 100 includes an electronic unit, including a pulse/signal generators 102 (current or voltage source) and a controller (programmed microcontroller) 106, connected to an electrodes' arrangement (including one or more electrodes' units) 104 of uncoupled electrodes. The electrodes are driven by the controller 106 that activates them according to predetermined operational patterns, i.e., sequences, which can be synchronous, asynchronous, or sequential. Each electrodes' unit delivers pre-defined pulse trains.

The operational data of the electrodes' unit (e.g., the pulse train parameters managed by the controller 106), as well as configuration/design of the electrodes' arrangement, is optimized (personalized) data, e.g., defined per patient, according to a personal treatment planning/regiment that allows protecting specific regions in the brain (such as the brain stem or the hippocampus) from exposure to electrical fields above stimulation threshold.

To this end, the system 100 is associated with a control unit 110 which is configured to generate the optimized personalized data and communicate such data to the system 100.

The control unit 110 is a computerized system including data input and output utilities 110A, 110C, memory (not shown) and a data processor which includes an optimization utility 110B. The optimization utility (software package) receives input data comprising clinical, imaging, pharmacokinetic and electric data and provides (i) operational data (a set of instructions) to the controller to operate the electrodes' units, and (ii) personalized electrode design data with regards to electrodes (electrodes' array) to be operated, to implement a personalized treatment regimen on the specific subject.

It should be noted that the control unit 110 may be integral with the controller 106, or the controller may be configured for data communication with the control unit 110 via wires or wireless communication using any known suitable type of communication and protocols.

The personalized data may be defined once for a specific patient and stored in the memory of the control unit 110 or in a memory of the controller 110 and properly used during treatment sessions of the respective patients.

The electrodes' arrangement presents an array of electrodes operable to form a number of electrode units. The case may be such that the electrodes' arrangement itself need not be specifically designed for a specific patient, the electrodes are placed on a head-wearable support/structure, and an electrodes' array of active electrodes (i.e., the electrodes that are to be activated/operated to be involved in the treatment session) is selected in accordance with specific patient's prescription. Such array of active electrodes presents personalized design of electrodes' arrangement.

Alternatively, the system may include a plurality of electrodes of various sizes and/or shapes and a head-wearable structure/support defining a plurality of electrode sites enabling to select from said plurality of electrodes the desired electrodes' array in association with the locations/sites on the support structure for placement of the selected electrodes, in accordance with the data indicative of the personalized design of electrodes' arrangement.

Thus, the optimization utility receives at least one of the following inputs: MRI data, pharmacokinetics data of drugs intended to use, clinical data (including at least data about desired disruption area, undesired disruption areas) and electric data including at least one of the following parameters: current and voltage limitation, electric (or electromagnetic) fields threshold, electric properties of the head layers and boundary conditions. The input data may also include data about disruption area over the BAB, AB-ratio (at least the minimal possible value), areas where electric (or electromagnetic) fields should be avoided or minimized.

The activation parameters may be selected from several predefined treatment programs and/or may be chosen individually per patient.

The distance between the electrodes (to be used for mounting the electrodes on the wearable electrode support structure, or to be used for selecting the electrodes to be activated) may be determined by the optimization utility, or may be predefined data on the already assembled support structure carrying the electrodes.

The microcontroller 106 distributes the activation parameters to the signal generator which operates the electrodes. A separate set of instructions may be delivered with respect to each electrode unit.

The output to the microcontroller 106 may be in the form of a set of instructions per electrode unit including pulse amplitude, pulse width, number of pulses and frequency.

Each electrode unit may be composed of at least two electrodes, one ground electrode and one energized electrode; or of several ground electrodes coupled with a single energized electrode; or of a single ground electrode and several energized electrodes; or of several ground and energized electrodes arranged in similar or different arrays.

The electrodes may be connected to one electric source or multiple separate source of the pulse generator.

The electrodes may be configured as coils and the generators activate the coils in order to produce electromagnetic fields on the BAB, sufficient for inducing BAB disruption.

It should thus be understood that the BAB disruption field may be electric field or electromagnetic field. In the description below the term “electric field” is used, but this term should be interpreted broadly covering also electric field having electromagnetic field components.

It should also be noted that the BAB disruption system includes or utilizes a head wearable support/structure (not shown here) which typically defines a plurality of electrodes' sites for a plurality of electrodes. When electrodes of the electrodes' arrangement 104 (at least one electrode unit) are mounted on such support, the electronic unit 102 independently operates each electrode unit to generate the pulsed electric field. The electrodes of each electrode unit have selected optimized geometrical and position parameters, and the controller 106 is configured to manage the operation of each electrode unit to generate the pulsed electric field of selected optimized electric parameters, such that the pulsed electric field within a target BAB region has a characteristic field profile and a characteristic field value which is above a predetermined BAB disruption threshold for a given pulse duration, and satisfies a condition of an optimized AB-ratio. As will be described below, the AB-ratio is a ratio between percentage of the target BAB region exposed to the pulsed electric field above the BAB threshold and percentage of brain tissues exposed to the electric field of a value above a predetermined brain threshold for the given pulse duration. The optimized AB-ratio may be of about 0.1 or higher.

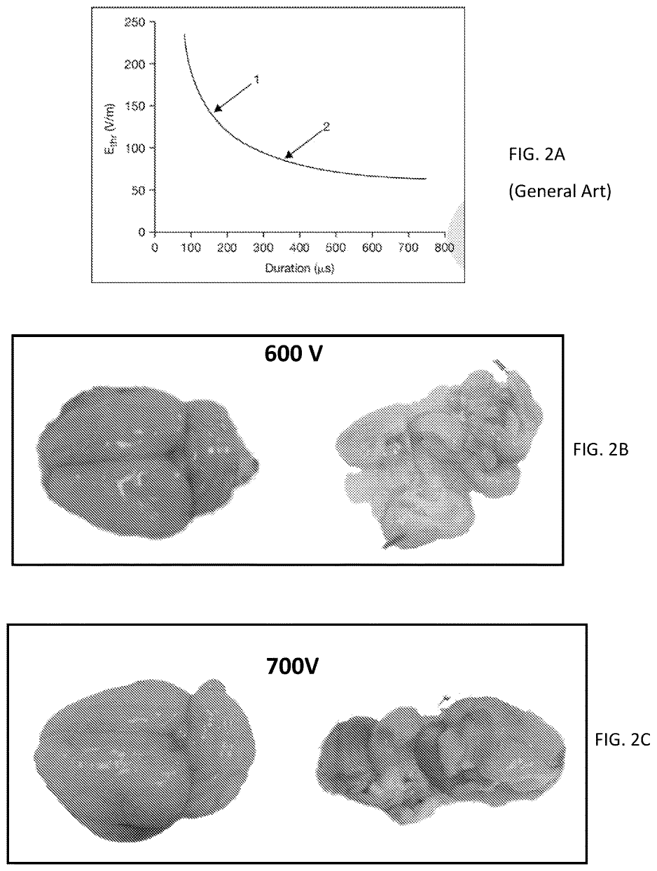

FIG. 2A exemplifies a strength duration curve for transcranial magnetic stimulation. As shown, using shorter pulses increases the neuronal stimulation threshold exponentially.

The system 100 may be configured as a kit for use by a medical team or for “home use” by a patient. In both cases, such kit includes an electronic unit including a pulse/signal generators 102 with the controller 106, a head-wearable support structure with the electrodes' arrangement 104 mounted on the respective electrode sites or in the form of a plurality of electrodes mountable on the prescribed sites.

Considering the kit to be used by a medical team to perform personalized treatment sessions on each patient, the respective patient's personalized data may be previously created and stored in association with the respective patient's ID in the memory of the controller 106 or in the memory of the external control unit 110 and accessible by the controller 106 via data communication. The personalized electrodes' array can be created by activation of the selected electrodes from the plurality of electrodes mounted on the wearable support structure using the personalized electrodes' arrangement design data, or can be created by mounting a corresponding sub-set from the plurality of the electrodes on the corresponding sites of the wearable support structure.

It should be noted that the personalized data, in relation to both the personalized electrodes' arrangement design data and the operational data of such electrodes' arrangement, is provided by the optimization procedure mentioned above and described more specifically further below. The personalized data may be patient-specific or may be population-specific, i.e., the common personalized data for a group of patients having similar patient-related data/parameters.

Considering the kit for “home use” by a patient, the patient's personalized data is previously created (using the optimization procedure) and stored in the memory of the electronic unit (e.g., in the controller 106) which the patient receives together with an electrode cap and the personalized electrodes' arrangement 104. The latter may be supplied while being mounted on the respective electrode sites of the support structure or as a separate set of electrodes allowing the patient to mount the electrodes on the predefined sites of the electrode support structure. In the latter case, the respective location data (matching identification data) can be properly provided on the electrodes and on the sites (locations) of the electrode support structure. The optimized data includes geometric and position data of the electrodes (i.e., electrodes' sizes, relative accommodation defining the electrode unit(s) and a distance between the electrodes of the unit and a distance between the units) and the electric data about the electric signal activation of the electrodes including the time pattern of the activating signal and their amplitudes. This optimized data defines the electric field profile and value which in turn defines the AB-ratio.

As described above, the optimized electrodes' arrangement to be involved in the treatment may be designed per patient, and such electrodes' arrangement can be properly assembled or may be formed by activation of the selected electrodes from a pre-existing plurality of electrodes. The electrodes of the electrodes' arrangement are driven by the programmed microcontroller 106 which activates them according to sequences that can be synchronous, asynchronous or sequential.

Thus, the control unit 110 is configured and operable to analyze the image data (typically MRI/CT) of the specific patient and determine the optimal AB-ratio with respect to a selected one or more BAB portions to be treated (i.e., disease-related data, drug-related data), as well as regions to be prevented from being affected by the electric field. Based on the optimal AB-ratio, the control unit provides the optimized personalized electric and geometric and position data for configuring and operating the electrodes' arrangement on the electrode support structure.

The optimization procedure is aimed at providing optimized electric field parameters for treatment of a selected BAB region for inducing BAB disruption while avoiding any BBB disruption. The electric field penetration to the brain is minimized to minimize neuronal stimulation through the brain or avoid neuronal stimulation in specific brain areas. The electric field penetration to the brain is limited based on the predefined optimized AB-ratio.

The BAB disruption may be combined with systemic drug administered to the patient in order to increase the drug concentration in the CSF. The BAB disruption may be intended to increase the clearance of substances from the CSF.

The inventors have investigated the applied voltage required to achieve a significant BAB disruption without BBB disruption using a rat model. Rats were injected with Evans Blue dye, a marker for BBB disruption and treated with a pulse train at different pulse amplitudes (100 pulses, 50 μs pulse duration at 4 Hz, pulse amplitude 200V-700V). In this connection, reference is made to FIGS. 2B and 2C show these experimental results. FIG. 2B shows rat examples of rat brains treated with PEGs at 600V and injected with Evans Blue dye, and FIG. 2C shows examples of rat brains treated with PEGs at 700V and injected with Evans Blue dye. These results demonstrate that below 700V (100 pulses, pulse duration of 50 μs at 4 Hz) no BBB disruption occurs and that the treatment is localized to the arachnoid barrier.

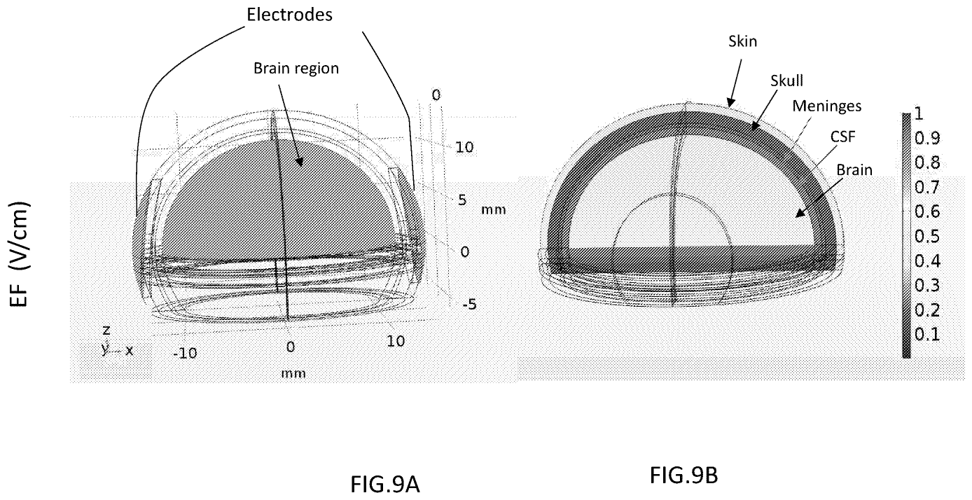

In these experiments, the electrodes setup configuration and operation similar to those of FIG. 9A can be used (i.e., the BAB area exposure to electric fields above 20 V/cm using 2 plate round electrodes with 1.2 cm diameter at 3 cm electrodes gap). The experiments show that for given pulse parameters it is possible to achieve isolated BAB disruption with the described electrodes setup.

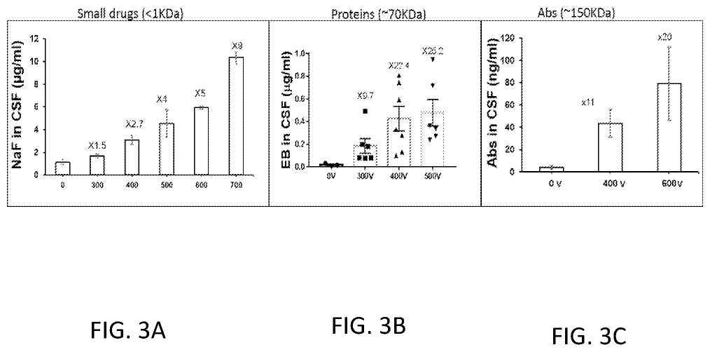

Reference is made to FIGS. 3A to 3C showing the results of an experiment conducted by the inventors where BAB disruption was achieved by applying 100 pulses (50 μs pulse duration at 4 Hz-total 25 sec) to the head of naïve rats. CSF was extracted from the foramen magna 1 h post treatment and concentration of different size molecules was quantified using plate reader. The figures show concentrations of Sodium Fluorescein (NaF)—FIG. 3A, Evans Blue (EB) tied to serum albumin—FIG. 3B, and Immunoglobulin G (IgG)—FIG. 3C in the CSF after application of PEFs (100 pulses, pulse duration of 50 μs at 4 Hz with different pulse amplitudes). The results demonstrate a significant increase in the concentration of the different molecules (with significant advantage to large molecules) as a function of applied voltage.

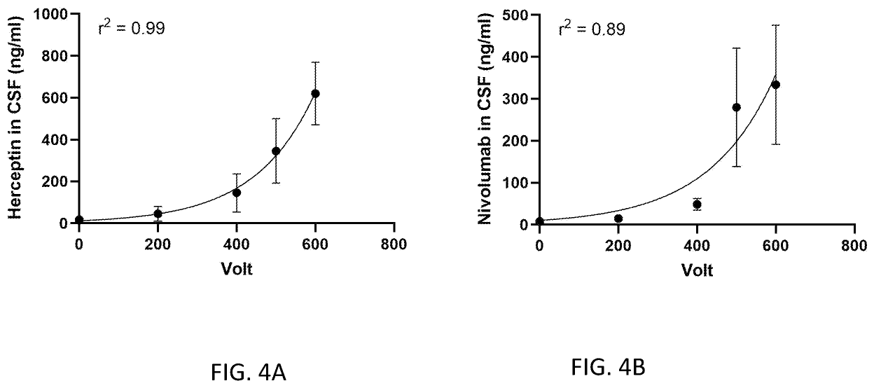

Reference is made to FIGS. 4A and 4B which show experimental results in rat model demonstrating voltage dependent effect of PEFs on the concentration of two monoclonal antibodies (mAb) based drugs, Nivolumab (Opdivo, nivolumab, concentrate for solution for infusion, 10 mg/ml, Bristol Myers Squibb) and Herceptin (Herceptin 440 mg IV (for IV infusion), Trastuzumab, Roche). The concentration of drugs in the CSF was studied using specific ELISA kits (Nivolumab (mAb-based) ELISA. Cat. No. TM09029, IBL and Trastuzumab (mAb-based) ELISA. Cat. No. TM09014, IBL). The results demonstrate a significant voltage dependent increase in drug concentration in the CSF.

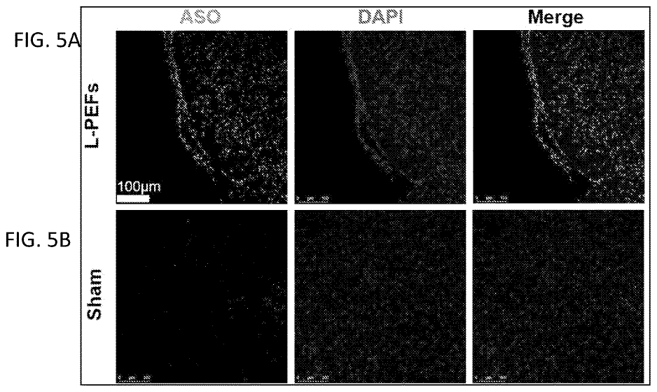

Antisense oligonucleotides (ASOs) were shown to distribute in the brain after injection to the CSF. This is shown in FIGS. 5A and 5B for representative cortices samples of L-PEFs (FIG. 5A) and Sham treated mice (FIG. 5B). The figures show PEFs treatment for BAB disruption enabling the penetration of significant amount of ASOs into mice brain from the CSF. More specifically, the figures demonstrate that IV injection of ASOs (Malat1-targeting ASO, 25 mg/kg.) in combination with 100 pulses (50 μs pulse duration at 4 Hz-total 25 sec) at 400V and the electrodes setup of FIG. 9A can also induce significant brain distribution of ASOs.

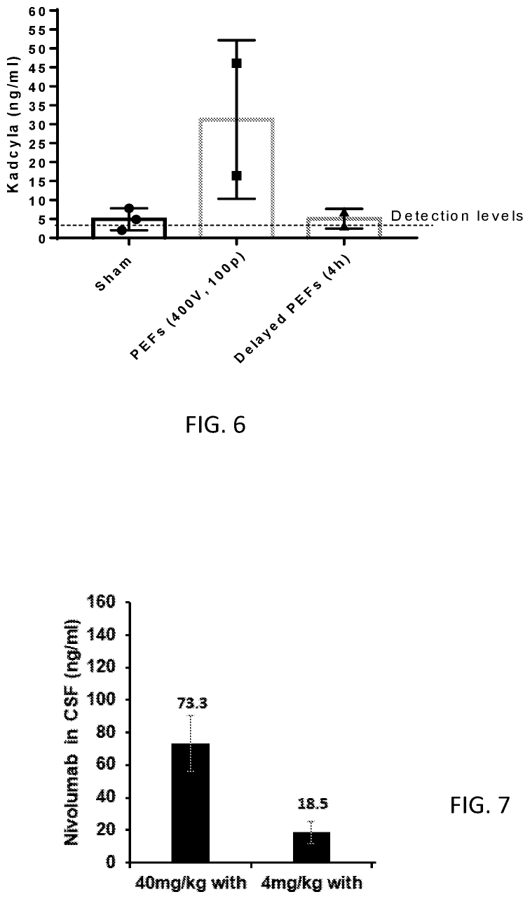

FIG. 6 shows the reversibility of the BAB disruption. In this experiment, Kadcyla Kadcyla, Trastuzumab emtansine, powder for concentrate for solution for infusion, 100 mg, Roche), an mAb-drug conjugate, was injected either immediately post PEFs treatment or 4 hours post treatment. The results demonstrate that at 4 hours, the BAB was recovered (I.E., the BAB is closed similar to the pre-treatment condition) and no increase in the drug concentration was found in the CSF.

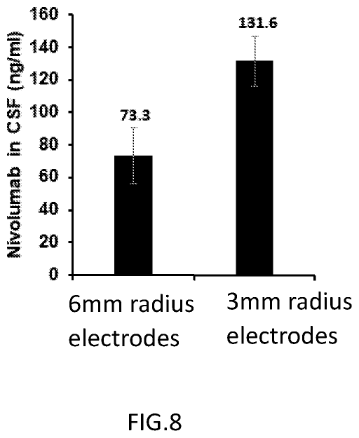

FIGS. 7 and 8 exemplify the treatment results as function of the drug dose and electrodes' size, respectively. More specifically, FIG. 7 demonstrates the effect of IV drug concentration on the concentration of CSF. Increasing the IV drug concentration ×10 resulted in ×4 increase in the CSF concentration using the same treatment parameters. FIG. 8 shows that reducing the electrodes radius by half while fixing all other treatment parameters, resulted in an increase in drug concentration in the CSF. This is due to electrode surface area being inversely proportional to current flow, thus reducing electrode size results in higher electric field between the electrodes.

Reference is made to FIGS. 9A and 9B, where FIG. 9A shows a geometrical 3D model of the rat head including electrodes location (6 mm radius at a distance of 30 mm) and layers build using COMSOL Multiphysics based on rat head dimensions obtained form the literature and from measurements of the layers using MRI, and FIG. 9B shows one slice of the model with the layers (skin, skull, meninges, CSF and brain) and the electric conductivity of each layer. Each slice was attributed with electrical properties from the literature including electric conductivity and permittivity.

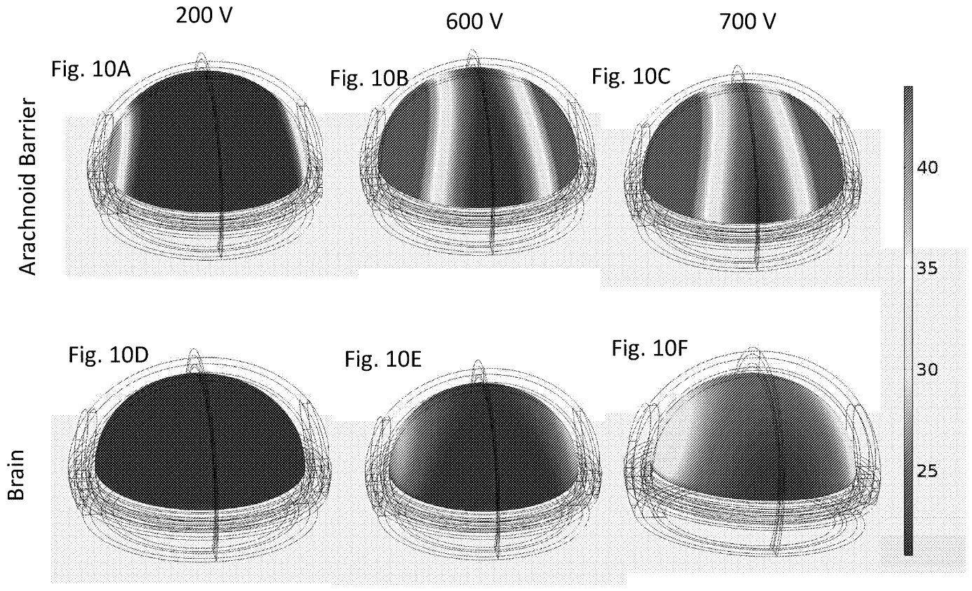

The results of the simulation for 6 mm radius electrodes with 30 mm distance are presented in FIGS. 10A-10F. FIGS. 10A-10C show examples of the electric field distribution on the BAB obtained with the electrodes setup shown in FIG. 9A and electric conductivities shown in FIG. 9B. FIG. 10A shows the results for applying 200V, FIG. 10B for 600V and FIG. 10C for 700V. FIGS. 10D-10F show the equivalent electric field over the brain parenchyma for each applied voltage. FIG. 10D shows the results for applying 200V, FIG. 10E for 600V and FIG. 10F for 700V. It should be noted that BBB disruption is only achieved from 700 V, thus the maximal electric field in FIGS. 10D-10E is below BBB disruption threshold.

Table 1 below shows the current and maximal electric field on the BAB and brain for different applied voltages.

TABLE 1

| Voltage | Current | Max | Max |
|---|---|---|---|
| (V) | (A) | EF BAB | EF brain |
| 200 | 0.28 | 44.42 | 11.80 |
| 400 | 0.56 | 88.84 | 23.61 |
| 500 | 0.70 | 111.05 | 29.51 |
| 600 | 0.83 | 133.27 | 35.41 |
| 700 | 0.97 | 155.48 | 41.32 |

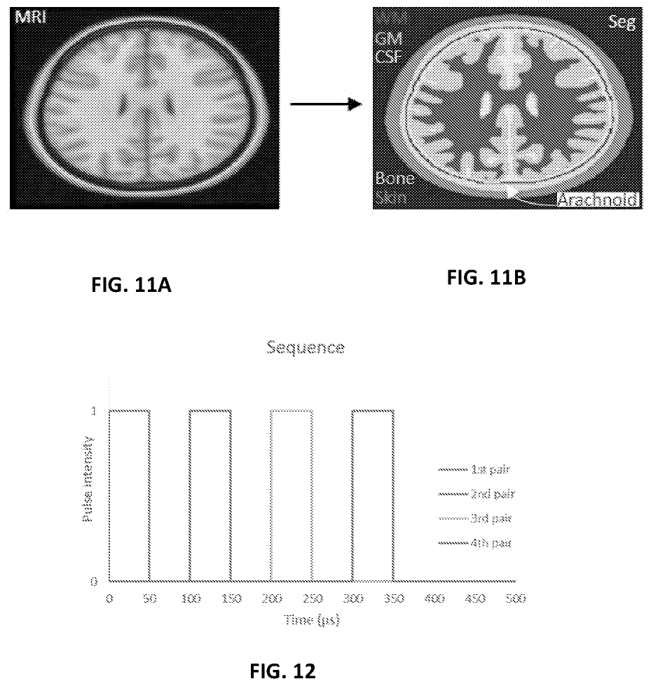

The distribution of electric fields in the different tissues of a human head were simulated using the ROAST (Realistic volumetric-Approach-based Simulator for Transcranial electric stimulation) simulation tool. First, the tissues were segmented using the SPM (Statistical Parametric Mapping) tool (software package designed for the analysis of brain imaging data sequences). As all brain segmentation tools exclude the meninges from the segmentation, the arachnoid tissue was defined as a 1 voxel shell extracted from the external surface of the segmented CSF. This is illustrated in (FIGS. 11A and 11B). After segmentation, different locations, sizes and distances between electrodes were modeled.

The technique of the present disclosure is aimed at and provides for optimizing the configuration and operation of the electrodes' arrangement in order to obtain BAB disruption (BABd) in significant BAB portions by inducing PEFs, above a given threshold, on the ABB, while, as much as possible, sparing the brain from exposure to the electric fields.

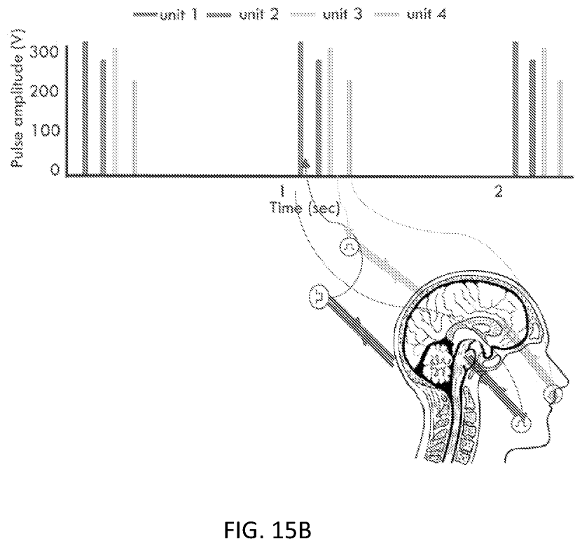

As described above, the system includes pulse generators (current or voltage source) connected to several units of electrodes. Such units of electrodes arranged along patient's head are exemplified in FIG. 15A, as will be described more specifically further below. The electrodes can be driven by a programmed microcontroller that activates them according to sequences that can be synchronous, asynchronous or sequential. FIG. 12 shows a non-limiting example of an activation sequence of the electrodes. These are established per patient, according to a personal treatment planning that allows protecting specific regions in the brain (such as the brain stem or the hippocampus) from exposure to electrical fields above stimulation threshold.

Each electrode unit (e.g., couple of electrodes) induces disruption in a specific BAB area (of x cm²), and each additional electrode unit (e.g., couple of electrodes) contributes an additional area of disruption. The size of the electrodes, the distance between them and the current are chosen in a manner enabling maximal BAB exposure and minimal brain exposure to electric fields. The frequency of activation is selected to be as low as possible as it was found to prevent the induction of seizures.

In some embodiments, treatment duration can be significantly shortened by placing multiple sets of electrode units on the head prior to treatment, and activating the electrode units in a synchronized manner, where the order to the units activated is chosen by their distance from each other, so that electrodes activated in close timing are relatively distant from each other. This is chosen in order to avoid stimulating large brain region at any given time. This way, if the treatment duration in any given location, applied by one couple of electrodes, is x min, the total treatment duration is still x min.

In some embodiments, the amplitude on each unit of electrodes is set according to the thickness of the skull near the electrodes' location, so that in regions where the skull is thinner, lower amplitudes are chosen in order to get electric field penetration similar to that in regions where the skull is thick.

The location of the electrodes can be set in an attempt to avoid stimulation of regions near tumors as tumors may lower the threshold for seizures, which is to be avoided. Also, the location of the electrodes can be set in an attempt to avoid non-intact regions in the skull (such as post op regions) in order to avoid penetration of high fields into the brain.

For example, 10-100 μs monopolar/bipolar pulses can be applied at a frequency of 0.5-100 Hz. The number of pulses for each electrodes unit can be in a range of 1-1000. As applying electric fields above a certain threshold to the brain may cause activation of neurons, the goal is to achieve as large as possible BAB disruption while minimizing brain activation. It has been reported that cortical motor neurons might be activated at electric fields above 0.45 V/cm when 300 μs pulses are applied. Strength duration curves (for transcranial magnetic stimulation) reveal that using shorter pulses increases the threshold exponentially.

In order to determine an optimal treatment protocol, a BRAIN threshold is set, for example 0.5 V/cm (chosen in this case for a maximal pulse duration of 100 μs to maximize safety), and a BAB threshold is set (meaning that above this threshold the BAB is disrupted), for example 20 V/cm. The optimal electrodes' configuration can be determined by optimizing the BA-ratio being a ratio between the percentage of BAB exposed to fields above the BAB threshold (i.e. ABd, “% ABd”) and the percentage of brain tissues exposed to fields above the BRAIN threshold (“% brain”):

$$AB - ratio = \% ABd / \% brain$$

As described above, the geometrical and position data of the electrodes' arrangement and the electric parameters/conditions of the electrodes' operation are selected to provide the desired AB-ratio to enable “surface treatment” of the arachnoid region (outer surface of the brain), providing effective BABd, while avoiding BBB disruption.

The electrical parameters of each electrode unit, having optimized geometrical and position parameters, may for example be as follows: electric current of 10 Amp-2 Amp (matching voltage depends on the impedance of the system and patient head) or voltage 10-2000V (matching current depends on the impedance of the system and patient head) with pulse duration in a range of 500 ns-10 ms, number of pulses 1-1000 of frequency of 0.5 Hz-1000 Hz. The number of electrode units may be in a range of 1-1000. The AB-ratio may be of 0.1 or higher. The electrodes may be of the following types: microelectrodes' array, surface electrodes, screw electrode or coils. The surface electrodes diameter may be about 0.5 mm-1.5 cm.

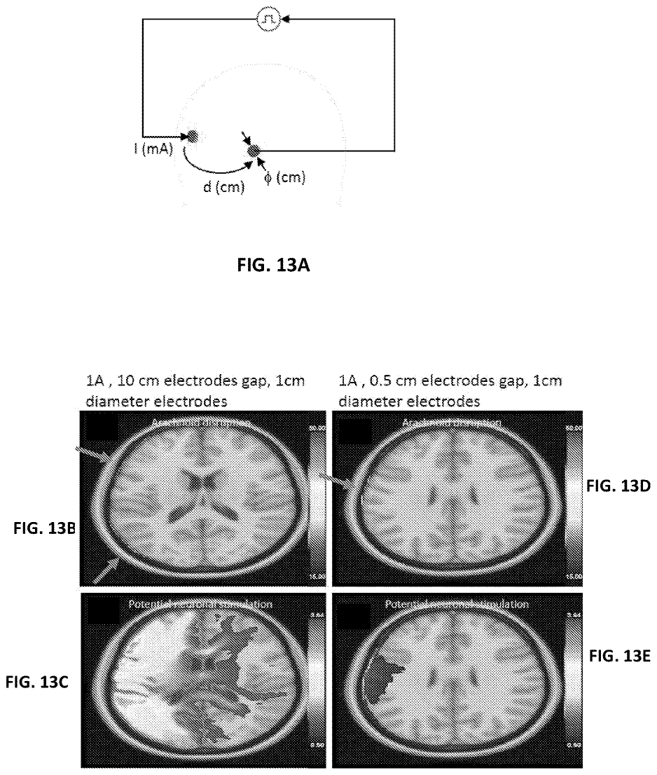

FIG. 13A shows schematically the parameters of electrodes' units that are optimized using the technique of the present disclosure. The diameter of the electrodes (φ cm), the distance between the center of the electrodes (d cm) and the applied current (I mA) (can be similarly performed with voltage) are examples of parameters that can be used for maximizing this ratio. The inventors have conducted human simulations of the electric field distribution on the BAB and brain parenchyma using various configurations and operational parameters of the electrodes' arrangements including: electrodes' arrangements including 1-20 electrode units, where each electrode unit can be operable with electric currents up to 0.4 Amp-1 Amp was used (matching voltage depends on the impedance of the system and patient head) or voltage of 400V-1000V (matching current depends on the impedance of the system and patient head), pulse duration 1-100 micro second, number of pulses 10-500, frequency 0.5-50 Hz.

FIGS. 13B to 13E show results of the human simulation of the electric field distribution on the BAB and brain parenchyma, wherein FIG. 13B shows the BAB area exposed to electric fields above 20 V/cm (estimated disruption threshold) using 2 plate round electrodes with 1 cm diameter at 10 cm electrodes gap with applied current of 1 A. FIG. 13C shows the electric field distribution above stimulation level in the brain using the electrode configuration of FIG. 13B. FIG. 13D shows the BAB area exposed to electric fields above 20 V/cm (the estimated BBB disruption threshold) using 2 plate round electrodes with 1 cm diameter at 0.5 cm electrodes gap with applied current of 1 A. FIG. 13E shows the electric field above stimulation level in the brain using the electrode configuration of FIG. 13D.

In the simulation examples shown in FIGS. 13B-13E, a preferred electrode setup would be 2 small flat/round electrodes (0.5 or 1 cm diameter) placed close to each other (distance between their centers 1.4 cm). This setup enables a relatively large ratio (~0.2), inducing disruption in ~0.5% of the BAB surface area (equivalent to ~4 cm² in the model) with a relatively low brain exposure (only ~2.7% of the brain exposed to fields above 0.5 V/cm).

Table 2 below summarizes the results of the simulation for a given current amplitude of 1 Amp, for several electrode sizes and distances between electrodes.

TABLE 2

| | Dist. | Dist. | | | |
|---|---|---|---|---|---|
| Electrode | between | between | | | |
| diameter | centers | edges | | | |
| (cm) | (cm) | (cm) | % AB | % brain | Ratio |
| 0.5 | 1.4 | 0.9 | 0.00561046 | 0.02703405 | 0.207533106 |
| 0.5 | 2.8 | 2.3 | 0.014190474 | 0.119848333 | 0.118403599 |
| 0.5 | 5.4 | 4.9 | 0.026655555 | 0.306600384 | 0.08693908 |
| 0.5 | 7.9 | 7.4 | 0.037113547 | 0.480581777 | 0.077226289 |
| 0.5 | 10.2 | 9.7 | 0.044167703 | 0.685570102 | 0.064424781 |
| 0.5 | 12.1 | 11.6 | 0.046421277 | 0.859318319 | 0.05402105 |
| 0.5 | 13.7 | 13.2 | 0.043862532 | 0.956197112 | 0.045871852 |
| 1 | 1.4 | 0.4 | 0.005000117 | 0.025255485 | 0.197981444 |
| 1 | 2.8 | 1.8 | 0.013932252 | 0.119664391 | 0.116427718 |
| 1 | 5.4 | 4.4 | 0.027395009 | 0.310266921 | 0.088294972 |
| 1 | 7.9 | 6.9 | 0.036855325 | 0.482075881 | 0.076451295 |
| 1 | 10.2 | 9.2 | 0.044202916 | 0.687406106 | 0.064303932 |
| 1 | 12.1 | 11.1 | 0.045693561 | 0.860261964 | 0.053115868 |
| 1 | 13.7 | 12.7 | 0.043369563 | 0.954506758 | 0.045436622 |
| 5 | 5.4 | 0.4 | 0.018192915 | 0.253772014 | 0.071689999 |
| 5 | 7.9 | 2.9 | 0.032089955 | 0.455140299 | 0.070505634 |
| 5 | 10.2 | 5.2 | 0.031913895 | 0.467593672 | 0.06825134 |
| 5 | 12.1 | 7.1 | 0.04226625 | 0.845492559 | 0.049990092 |
| 5 | 13.7 | 8.7 | 0.041080777 | 0.947663967 | 0.043349518 |
| 5 | 5.4 | 0.4 | 0.018192915 | 0.253772014 | 0.071689999 |
| 5 | 7.9 | 2.9 | 0.032089955 | 0.455140299 | 0.070505634 |

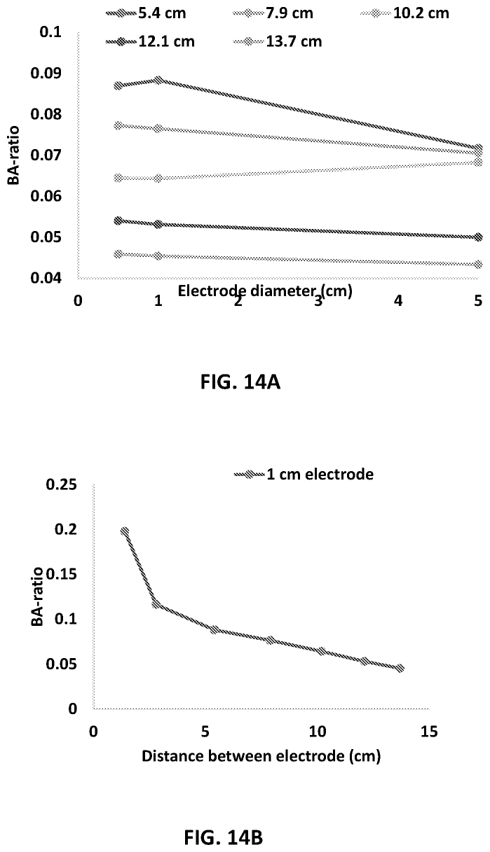

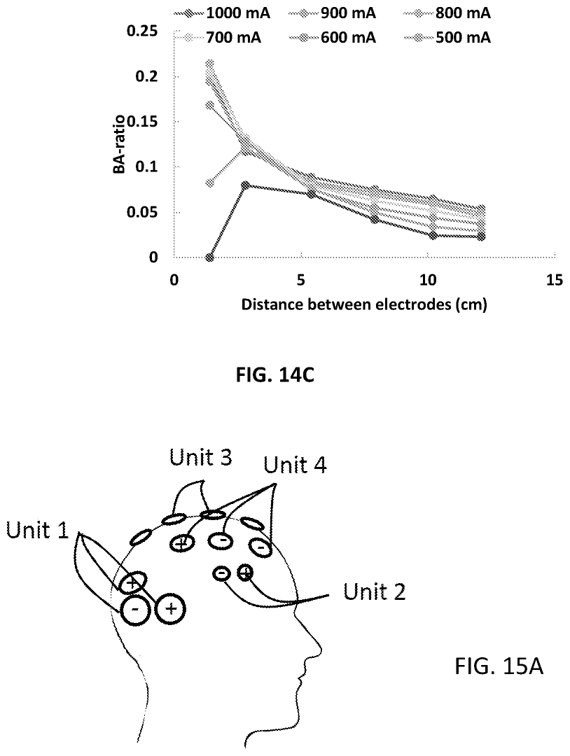

Reference is made to FIGS. 14A to 14C, where FIG. 14A shows the AB-ratio for the case that the BAB disruption threshold is set to 20 V/cm and neuronal stimulation threshold is set to 0.5 V/cm as a function of electrode diameter for different distances between the electrodes' centers and different electrodes diameters for a given electric current of 1 Amp; FIG. 14B shows the AB-ratio for a given current amplitude (1 Amp) and a given electrode size (1 cm) for different electrode gaps (d); and FIG. 14C shows the AB-ratio for different electrodes gaps (d) and applied currents (mA) with a given size of electrodes.

For a given distance between the electrodes centers and a given current (1 Amp), the size of the electrodes did not change significantly the AB-ratio, as shown in FIG. 14A.

FIG. 14B shows that for a given current amplitude (1 Amp) and a given electrode size (1 cm), the AB-ratio improved mostly with smaller distances between the electrode centers. Using smaller electrodes allows smaller distances between centers.

Table 3 below summarizes the results of the simulation for 1 cm diameter electrodes for several current intensities and distances between the electrodes.

TABLE 3

| | | Distance between electrode centers (cm) | | | | | |
|---|---|---|---|---|---|---|---|
| Ratio | | 1.4 | 2.8 | 5.4 | 7.9 | 10.2 | 12.1 |
| Cur [symbol] | 400 | 0 | 0.079654433 | 0.069899 | 0.042057 | 0.024357 | 0.02301 |
| | 500 | 0.082198104 | 0.121453903 | 0.076245 | 0.049785 | 0.03399 | 0.029403 |
| | 600 | 0.168260082 | 0.1282132 | 0.075008 | 0.054917 | 0.043775 | 0.037187 |
| | 700 | 0.20409942 | 0.132405573 | 0.079215 | 0.062805 | 0.051483 | 0.042379 |
| | 800 | 0.214244234 | 0.127967749 | 0.081056 | 0.068049 | 0.058656 | 0.045409 |
| | 900 | 0.194449788 | 0.12094259 | 0.08385 | 0.071861 | 0.060785 | 0.049474 |
| | 1000 | 0.202555996 | 0.117008628 | 0.088799 | 0.075163 | 0.064781 | 0.053626 |

[symbol]

indicates data missing or illegible when filed

For a given size of electrodes, the smallest distance between the electrodes provides the largest AB-ratio. Larger distances need to be considered for lower currents (larger distance between the electrodes allows increased penetration which may compensate for the lower penetration caused by the lower currents). According to the simulation, the best compromise between distance and current can be computed for each treatment, as shown in FIG. 14D.

Reference is made to FIGS. 15A-15B exemplifying an activation sequence of the electrodes, enabling low brain exposure, short treatment duration and accumulative large ABd area. Assuming 50 μs pulse width every second (1 Hz), up to 20000 (= 1/50e⁻⁶) pairs of electrodes can be activated successively between 2 consecutive pulses without increasing the treatment duration, by taking advantage of the small duty cycle of the activation profile of each pair.

Electrode activation schemes can be designed to successively expose accumulatively most of the AB surface (where each unit of electrodes is pulsed at 1 Hz), keeping low electric field exposure in the brain/low currents per pulse, and avoiding positions that could expose specific areas of the brain (such as the brain stem and the hippocampus).

Diseases that involve the brain and meninges are significantly difficult to treat, mainly due to the BBB and BCSFB including the BAB that prevent the penetration of drugs to the pathological area. In the last years, great efforts have been made to find methods to transiently disrupt the BBB. Nevertheless, the BAB is generally overlooked. On top of the potential benefits of BAB disruption for meningeal disease, CSF circulation is now increasingly viewed as a viable pathway to deliver certain therapeutics deeper into brain tissues.

The present disclosure provides a technique enabling increase of drug concentration in the CSF using pulsed electric fields. Existing methods for inducing electric fields in the brain using contract electrodes focus on penetrating the brain. The technique of the present disclosure provides focusing the electric fields on the BAB—i.e. a thin surface surrounding the brain, with emphasis on minimizing brain exposure to electric fields in order to achieve safe BAB disruption. Additionally, the novel technique of the disclosure uses unsynchronized pulse application to minimize treatment duration.

FIG. 15A schematically illustrates electrodes configuration for applying electric fields to induce BAB disruption in a treatment region on the surface of the arachnoid of a subject, in which 4 units of electrodes are placed on the skin of the subject above the treatment region. For each electrodes' unit, 1-4, the electrodes diameter and distance between the electrodes may vary as well as the number of electrodes. For example, in unit 1, two electrodes are positive, and one is negative, in unit 2 one electrode is negative and one positive, in unit 3 one electrode is negative and one positive as I unit 2 but the diameter is larger as well as the distance between the electrodes, and in unit 4 one electrode is positive and two electrodes are negative.

FIG. 15B shows an example of phased delay synchronous activation of 4 electrodes units. In this example each of the 4 units of electrodes is activated at a delay of 100 μs from the previous unit, while all units are activated during the interval between the pulses of the other units. Using this activation method, each unit delivers a set number of pulses (for example 100 pulses) at a relatively low frequency (for example 1 Hz), only one unit is activated at any given moment, keeping brain exposure volume to a minimum while BAB is disrupted in several regions during 100 second. If the 4 units were activated serially the treatment would last 400 seconds. In this manner it is possible to perform up to 10,000 treatments in 100 seconds.

The following is the example of the treatment protocol for using the technique of the present disclosure for blood-brain barrier (specifically blood-arachnoid barrier) disruption using barrier disrupting fields (BDF) in subjects with leptomeningeal disease (LMD), aimed at assessing the performance of BDF in opening the blood-brain barrier (BBB), specifically blood-arachnoid barrier (BAB) in subjects.

The primary endpoint is the change from baseline (pre BDF treatment) in concentration of Nivolumab or Trastuzumab (depending on the type of primary tumor) antibodies in the cerebrospinal fluid (CSF). The secondary endpoint includes the change from baseline in biomarkers in the blood and CSF; and the change from baseline in Quality of Life (QoL) questionnaires. The main safety endpoint is the overall incidence of Barrier Disrupting Fields (BDF) treatment related Adverse Events (AEs) and Serious Adverse Events (SAEs), with severity graded according to CTCAE v5.0 criteria. Other safety endpoints include all AEs related and unrelated to the study treatment, vital signs, blood, and urine tests.

The study population can include up to 15 adult patients with melanoma or lung or HER2+ breast cancer candidates for treatment of leptomeningeal spread. The study sample size can be up to 15 treatments. Each subject can receive up to 3 treatments. Therefore, the study population can range from 5 to 15 subjects.

The study procedure can be as follows:
Subjects with leptomeningeal spread of melanoma, lung or HER2+ breast cancer who meet eligibility criteria will be enrolled;
Subjects will undergo up to 3 BDFs treatments, each performed 2-3 hours after their IV antibody treatment.
Baseline brain MRI will be acquired prior to any study procedure, unless MRI was acquired prior to enrollment and up to 2 weeks prior to BDF treatment, and repeated 4-7 weeks after the first treatment—during the 3^{rd} study treatment hospitalization (the day following BDF treatment).

The procedure steps can be as follows:
Enrolled subjects are asked to provide or undergo tests that are not available from the last 3 weeks including MRI, echo cardiography, ECG, and blood test.
The day prior to treatment, patients undergo a physician assessment, complete blood test and chemistry.
Subjects are admitted to the oncology department in the morning of treatment and following physician examination and lab tests are treated with IV infusion of study medication (Nivolumab or Trastuzumab). Melanoma and lung cancer subjects are treated by Nivolumab and HER2+ breast cancer subjects by Trastuzumab.
2-3 hours after the IV infusion, a lumbar drain is placed. CSF and blood samples are taken as baseline.
BDFs treatment is provided for up to 12 min under anesthesia.
CSF (5 cc per sample) and blood samples are taken at 30 min, 60 min, 90 min, 120 min, and 7-8 hours (just prior to lumbar drain removal) post BDFs. Each subject's baseline serves as a control for all other measurements.
The subject is discharged the next morning and followed for up to 2-3 weeks by a weekly phone call from the research coordinator.
Subjects return for a second and third treatment 2-3 weeks following the previous one.

BDF treatment is as follows: Treatment can be provided in ECT conditions; subjects who are not receiving anti-epileptic treatment receive anti-epileptic medication prior to BDF treatment. Standard anesthesia monitoring is performed including ECG, noninvasive blood pressure measurement, pulse oximetry, capnography and EEG. The BDF treatment can be provided under up to 12 min of anesthesia provided by an anesthesiologist-Propofol, Midazolam and muscle relaxant, followed by 30 minutes of hemodynamic and respiratory monitoring.

Steroid dosing is to be stable during the CSF sampling period. Also, it should be noted that all the time intervals of various processes/procedures of the treatment protocol may vary within ±20%.

1.-37. (canceled)

38. A blood-arachnoid barrier (BAB) disruption system comprising:
a head wearable support structure defining a plurality of electrode sites for a plurality of electrodes of an electrodes' arrangement, said electrodes arrangement comprising at least one independently operable electrode unit configured and operable to generate an electric field; and an electronic unit comprising at least one electric pulse generator and a controller configured and operable for managing operation of each of said at least one electrode unit to generate the electric field,
wherein each of said at least one electrode unit comprises the electrodes of selected optimized geometrical and position parameters, and the controller is configured and operable to manage the operation of each of said at least one electrode unit to generate the electric field being a pulsed electric field of selected optimized electric parameters, such that the electric field produced by the electrodes' arrangement within a target BAB region has a characteristic field profile and a characteristic field value being above a predetermined BAB disruption threshold for a given pulse duration, and satisfies a condition of an optimized AB-ratio, being a ratio between percentage of a BAB region exposed to the electric field above said BAB threshold and percentage of brain tissues exposed to the electric field of a value above a predetermined brain threshold for the given pulse duration, thereby avoiding BBB disruption by said electric field.

39. The BAB disruption system according to claim 38, wherein said optimized AB-ratio is at least 0.04.

40. The BAB disruption system according to claim 38, wherein said optimized geometrical and position parameters comprise electrode size for each electrode of the electrode unit, and a distance between centers of the electrodes of the electrode unit.

41. The BAB disruption system according to claim 38, wherein the electrodes' arrangement comprises the at least one electrode unit comprising at least two electrodes comprising at least one grounded electrode and at least one energized electrode.

42. The BAB disruption system according to claim 38, wherein the electrodes' arrangement comprises electrodes connectable to the same electric source or multiple separate sources of the electronic units.

43. The BAB disruption system according to claim 38, wherein said electrodes' arrangement comprises electrodes configured as coils, and said at least one electric pulse generator comprises an alternating electromagnetic source, said electric field comprising electromagnetic field components.

44. The BAB disruption system according to claim 38, wherein the electrodes' arrangement comprises electrodes of any of the following types: microelectrodes array, surface electrodes, screw electrode or coils.

45. The BAB disruption system according to claim 38, wherein said selected optimized electric parameters comprise parameters of the pulsed electric field comprising one or more of the following: amplitude, pulse width, pulse shape, pulse duration, number of pulses and frequency.

46. The BAB disruption system according to claim 38, wherein the electronic unit is configured to operate each of said at least one electrode unit with the following electric parameters: electric current in a range of 10 mAmp-2 Amp or voltage in a range of 10-2000V, pulse duration of in a range of 500 ns-10 ms, a number of pulses in a range of 1-1000, a frequency in a range of 0.5 Hz-1000 Hz, and a number N of the electrode units in a range of 1-1000.

47. The BAB disruption system according to claim 38, wherein said electrodes' arrangement is configured in accordance with an optimized treatment plan selected for a patient.

48. The BAB disruption system according to claim 38, wherein said electrodes' arrangement is defined by selected electrodes, from a plurality of electrodes mounted on said support structure, in accordance with an optimized treatment plan for a patient.

49. The BAB disruption system according to claim 38, wherein said selected optimized electric parameters comprise parameters defining electrodes' driving pattern.

50. The BAB disruption system according to claim 49, wherein said driving pattern corresponds to synchronous, asynchronous or sequential activation of the electrodes.

51. The BAB disruption system according to claim 38, wherein said electrodes' arrangement comprises a plurality of the independently operable electrode units, each forming a separately operable electric circuit.

52. The BAB disruption system according to claim 38, wherein the controller is configured and operable to communicate with a control unit to receive therefrom personalized operational data comprising data indicative of the selected optimized geometrical and position parameters of the electrodes to be activated for BAB disruption session, and the selected optimized electric parameters of said electrodes to be activated.

53. A control unit configured and operable for managing BAB disruption treatment performed by electric field application, the control unit being configured as a computerized system comprising data input and output utilities and a data processor comprising an optimization utility configured and operable to analyze input data indicative of static image data of a patient's brain region, and determine personalized operational data comprising data indicative of selected optimized geometrical and position parameters of a plurality of electrodes to be activated during a BAB disruption treatment of a target BAB region and selected optimized electric parameters of said electrodes being activated to generate a pulsed electric field, such that the pulsed electric field produced by the electrodes' arrangement has a characteristic field profile and a characteristic field value being above a predetermined BAB disruption threshold for a given pulse duration of said pulsed electric field, and satisfies a condition of an optimized AB-ratio between percentage of the target BAB region exposed to the electric field above said BAB threshold and percentage of brain tissues exposed to the electric field of a value above a predetermined brain threshold for the given pulse duration, thereby avoiding BBB disruption by said electric field.

54. The control unit according to claim 53, wherein said input data comprises at least one of the following: MRI data of a patient, pharmacokinetics data of drugs intended to use, clinical data, and electrical data.

55. The control unit according to claim 54, wherein the electrical data comprises at least one of the following parameters: current and voltage limitation, electric fields threshold, electric properties of head layers and boundary conditions.

56. The control unit according to claim 53, wherein the input data comprises data indicative of one or more of the following: disruption area over the BAB, AB-ratio, areas where electric fields are avoided or minimized.
